(19) United States
(12) Patent Application Publication
(10) Pub. No.: US 2007/0219582 A1
(43) Pub. Date: Sep. 20, 2007

(54) DEVICE FOR MANIPULATION AND PLACEMENT OF FLEXIBLE IMPLANTS

(75) Inventors: John E. Brunelle, Huntington Beach, CA (US); Thomas W. Sander, Gainesville, FL (US)

Correspondence Address: WILSON SONSINI GOODRICH & ROSATI, 650 PAGE MILL ROAD, PALO ALTO, CA, 94304-1050, US

(73) Assignee: Pegasus Biologics, Inc.

(21) Appl. No.: 11/276,934

(22) Filed: Mar. 17, 2006

(51) Int. Cl. A61B 17/00

(52) U.S. Cl. 606/207

(57) ABSTRACT

A novel surgical instrument for holding a flexible implant is provided, as is a novel flexible implant to be held by such instrument. The novel surgical instrument comprises flexible implant hooks. The flexible implant comprises one or more apertures, which are used to hang the flexible implant on the flexible implant hooks of the instrument. The instrument is adapted to hold the implant, deliver it to a tissue and hold it in place while a surgeon makes a surgical repair using the flexible implant.

### BACKGROUND OF THE INVENTION

The musculoskeletal system comprises numerous connective tissues that join muscle to bone (tendons) and bone to bone (ligaments). The body is dependent upon these connective tissues for proper operation of joints. From time to time, connective tissues may become injured, thereby reducing the quality of joint function. Numerous treatment options have been proposed for treatment of connective tissue tears. For example, surgical repair or replacement of connective tissues has been used for tendon repair in the shoulder, knee, hip and ankle. Arthroscopic, mini-open and full-open surgical approaches have been applied, with varying results, to the repair of tendons and ligaments in various types of joints. In some of such surgical procedures, the attachment between the bone and connective tissue is re-enforced (or, in the case of complete rupture, re-established) by means of sutures, staples and/or combinations of sutures and bone anchors. The goal of such re-enforcement is to provide mechanical support for the joint and its associated connective tissue, thereby restoring joint function in the near term and allowing for remodeling of the connective tissue, thereby strengthening the repaired tissue over the long term.

Allograft tissue prostheses are known and have been used to replace or reinforce a variety of bodily tissue. For example, gauze-reinforced keratinocyte sheets have been used to treat severe melanoma. The melanocytic nevus is removed and keratinocyte sheets are implanted over the resulting wounds. Other tissue prostheses, such as demineralized bone and decellularized collagen sponges have also been used with positive effects. Such tissue engineered products aid in healing by providing a substrate or matrix into which blastocytes, osteocytes, keratinocytes and other cytic cells can migrate and establish new living tissue within the repair site, thereby strengthening the repair.

Although it is considered desirable to use collagen implants for repair or reinforcement of connective tissues and other tissues, especially muculoskeletal tissues, it has been found to be difficult to apply such tissues to various surgical repairs. In the surgical repair of some connective tissues, such as tendons and ligaments, it is necessary to hold a reinforcing implant in contact with the connective tissue while the surgeon attaches the implant to the connective tissue and the combination of the implant and the connective tissue to the bone. The surgeon is, in many circumstances, required to hold the implant tissue in contact with the connective tissue with one hand while connecting the implant to the connective tissue, and the combination to the bone, with the other hand. Thus, the tissue implant is subject to movement and/or buckling during the procedure, with the result that the implant may not always make optimal contact with the connective tissue, the bone or both. While it is conceptually possible to have a surgical assistant hold the implant in contact with the connective tissue while the surgeon makes the necessary attachments, this alternative is unworkable in many surgical procedures, especially mini-open and arthroscopic surgery, where access is limited. Moreover, existing surgical instruments have proven to be inadequate for addressing this problem.

There is a need for a surgical instrument capable of holding and delivering a flexible implant in a surgical subject. There is also a need for a surgical instrument capable of holding, delivering and clamping in place a flexible implant. There is further a need for a flexible implant adapted to be held, delivered and clamped in place. There is further a need for surgical kits adapted to hold, deliver and clamp in place a flexible implant. There is moreover a need for surgical methods of implanting such flexible implants. These and other needs are met by embodiments of the present invention.

### SUMMARY OF THE INVENTION

The foregoing and further needs are met by embodiments of the invention, which provide a surgical instrument comprising a set of jaws in communication with an actuator. The set of jaws comprises at least one implant hook; and the actuator is adapted to open and close the jaws. The surgical instrument is adapted for holding a flexible implant of the invention, which is described in more detail below. It is further adapted to place the flexible implant within a body, to clamp it in place and to hold it in place while the operator attaches the implant within the body. The invention also provides a surgical implant kit comprising the surgical instrument described herein and a flexible implant of the invention. In some embodiments, the kit further comprises a sterile bag in which the flexible implant is contained separately from the surgical instrument and the sterile bag and surgical device are contained within a sterile package. In some preferred embodiments, the kit further comprises instructions for operation and/or storage of the instrument with the flexible implant. The invention also provides an implantation device comprising a surgical instrument of the invention and a flexible implant of the invention attached to the surgical instrument of the invention. The instrument and implant of the invention are as described in detail below.

The surgical instrument of the invention comprises a set of jaws that is opened and closed by an actuator. The set of jaws (together also referred to herein as the head) comprises two or more jaws, preferably two jaws, at least one of which is designed to hold the flexible implant on the instrument without the operator having to maintain tension on the instrument. The implant is held on the instrument with one or more components designed to receive a part of the implant and hold it firmly without the operator having to continuously apply force to the instrument. This allows the operator to manipulate the instrument and the implant within a body, to clamp the implant into place, and to secure the implant to a bodily tissue. The component designed to hold the flexible implant on the instrument is referred to herein as an “implant hook” or “hook.” As the term implies, the implant hook is adapted to allow a flexible implant to hang from the hook. In some embodiments, a hook is a tapered, curved or bent member extending from a jaw of the instrument. The taper, curve or bend may be in any plane (up, down or to either side). In some embodiments, the taper, curve or bend permits the instrument to hold the flexible implant without the operator having to maintain force on the instrument, thereby allowing the operator to manipulate and/or lock in place the instrument during a surgical procedure. In some embodiments, at least one hook is augmented with one or more additional features that facilitate maintaining the implant on the instrument during a surgical procedure, to assist it in grasping a bodily tissue during the surgical procedure, or to assist the operator by permitting further manipulations of the instrument and/or the bodily tissue during the procedure. These and additional features are discussed in greater detail below.

In accordance with the invention, the set of jaws comprises at least one implant hook. In some embodiments, the set of jaws comprises only one implant hook. In further embodiments, the set of jaws comprises two or more implant hooks. In some embodiments, the set of jaws comprises 2, 3, 4, 5, 6, 7, 8, 9, 10 or more implant hooks.

In addition to implant hooks, the instrument may include one or more additional features designed to assist it in maintaining the implant on the instrument during a surgical procedure, to assist it in grasping a bodily tissue during the surgical procedure, or to assist the operator by permitting further -manipulations of the instrument and/or the bodily tissue during the procedure. Such additional features can include one or more contact pads, fingers, grasping slits, suture or needle channels, hollow needles or puncture spikes.

In some embodiments, the set of jaws further comprises at least one contact pad. As their name implies, contact pads are portions of the jaws that are adapted to contact a bodily tissue during a surgical procedure. Such contact pads are located on the interior portion of the jaws. As used with reference to the jaws and parts of the jaws, the term interior indicates the space between the jaws. In some embodiments, the contact pads are textured to provide enhanced grip when the instrument is clamped to a bodily tissue in a surgical subject. In some embodiments, the contact pads may have other features, such as suture or needle channels, hollow needles or puncture spikes to assist the surgeon in connecting the implant to a bodily tissue. In some embodiments, the channels are apertures in the contact pad or other part of the jaw, which allow passage of a needle and/or a suture through the instrument and into the bodily tissue and/or implant on the other side of the instrument. In some embodiments, the hollow needles and puncture spikes are adapted to pierce the bodily tissue and/or the implant. In some embodiments, at least one of the jaws comprises one or more fingers and the contact pads are located near the ends of the fingers and in particular on the interior portion of the fingers. In some embodiments, the set of jaws comprises two or more contact pads. In some embodiments, the set of jaws comprises 2, 3, 4, 5, 6, 7, 8, 9, 10 or more contact pads.

In some embodiments, the set of jaws comprises at least one grasping slit. A grasping slit is an indentation in a part of a jaw, such as the end of a jaw and above an implant hook, that is adapted to receive at least a portion of a flexible implant, thereby grasping the implant firmly to allow for holding and manipulation of the flexible implant. In some embodiments, the set of jaws comprises only one grasping slit. In some embodiments, the set of jaws comprises 2, 3, 4, 5, 6, 7, 8, 9, 10 or more grasping slits. In some embodiments, the number of grasping slits equals the number of implant hooks.

The instrument is amenable to being adapted for use in a variety of surgical procedures. In some cases, it is necessary for the implant to be employed in bodily locations where it is desirable to have the head offset from the actuator, e.g. by tilting it up, down or to either side so that the actuator is disposed at a different angle than the implant. In some embodiments, this allows the implant to be placed and held in place in hard-to-reach body spaces. In some embodiments, this allows the operator to place the actuator out of the way of the field of view. In some embodiments, this allows the operator to place the actuator out of the way of the field of operation. In some embodiments, the set of jaws is disposed at an angle with respect to the actuator. For example, where the set of jaws is located at the end of a set of scissor-like arms, the set of jaws may be angled up, down, to one side or to the other with respect to the arm. In some embodiments, the angle of the set of jaws (also referred to herein as the “head”) is about 1 to about 135 degrees. In some particular embodiments, the angle of the head is about 30 to about 90 degrees. In other particular embodiments, the angle is about 5, about 10, about 15, about 20, about 25, about 30, about 35, about 40, about 45, about 50, about 65, about 70, about 75, about 80, about 85, about 90, about 95 or about 100 degrees.

As mentioned above, the surgical instrument of the invention possesses a set of jaws. In some embodiments, the surgical instrument comprises at least two jaws, each jaw comprising at least one finger. At least one of said fingers on at least one jaw has an implant hook thereon. As mentioned above, the fingers increase the length of the instrument, allowing the operator to actuate the device at a greater distance from the implant. The fingers may be bent or curved in any direction, independently or together, in order to aid them in navigating the particular bodily space for which the instrument is intended to be used. For example, in some embodiments, both fingers on each jaw are bent inward with respect to the jaw. (E.g. on a two-jaw instrument, one jaw being the upper and the other being the lower, the fingers on the upper jaw are bent downward and the fingers on the upper jaw are bent upward.) In some embodiments, both fingers are bent outward. In some embodiments, fingers on each jaw are bent or curved to the left or the right (looking down on the instrument with one jaw on top and the other jaw on the bottom). In some embodiments, each jaw comprises one finger only. In some embodiments, each jaw independently comprises two or more fingers. In some embodiments, each jaw independently comprises 2, 3, 4, 5 or more fingers. In some embodiments, each jaw has the same number of fingers. In some embodiments, each finger on one jaw is directly opposed by a finger on the other jaw. In some embodiments, the two jaws have different numbers of fingers. In some embodiments, at least one finger on each jaw is directly opposed by at least one finger on the other jaw. In some embodiments, at least one finger on one jaw is offset with respect to the fingers on the opposing jaw. In some embodiments, all fingers on each jaw are offset with respect to the fingers on the other jaw. In some embodiments, at least one finger comprises texturing (such as serration), a needle or suture channel, a hollow needle or a puncture spike. In some embodiments, at least one finger comprises a needle, a suture, a channel, a hollow needle or a puncture spike. In some embodiments, at least one finger comprises texturing. In some embodiments, at least one finger on each jaw comprises texturing. In some embodiments, each finger on at least one jaw comprises texturing. In some embodiments, each finger on one jaw only comprises texturing. In some embodiments, each finger on each jaw comprises texturing.

The device employs an actuator to open and close the jaws. The actuator may be any actuator known in the art for opening and closing jaw-like devices. Such actuators include scissor-like handles as well as thumb actuators. In some embodiments, the actuator further comprises a lock adapted to lock said jaws in a closed position. In some particular embodiments, the actuator comprises a set of handles or a thumb actuator. In some particular embodiments, the actuator is a set of handles. The handles may be straight or curved, and especially may be curved downward or upward. Thus, in some embodiments, the set of handles is curved. In some particular embodiments, the device is adapted for arthroscopic surgery.

As mentioned above, the jaws may include texturing to enhance the instrument's ability to grasp and/or hold in place a flexible implant. Thus, in some embodiments, the set of jaws comprises at least one textured grip. In some embodiments, the jaws comprise 1, 2, 3, 4, 5, 6, 7, 8, 9, 10 or more textured grips.

The foregoing and further needs are met by embodiments of the invention, which provide a flexible implant specifically designed to operate with the instrument of the invention. Thus, in some embodiments, the invention provides a flexible implant, comprising a flexible sheet having at least one aperture adapted to rest on an implant hook. In some embodiments, the flexible implant has one aperture only. In some embodiments, the flexible implant has from one to 10 or more apertures adapted to rest on implant hooks of a surgical instrument of the invention. In some embodiments, the flexible implant has two ends, at least one end having at least one aperture therein. In some embodiments, each end has at least one aperture therein. In some embodiments each end independently has 1, 2, 3, 4, 5 or more apertures therein. In some embodiments each end has the same number of apertures as the other. In some embodiments one end has a greater number of apertures than the other. In some embodiments the sheet of flexible material comprises a soft tissue or a flexible polymer. In some embodiments the sheet of flexible material is a soft tissue comprising a crosslinked mammalian tissue, such as crosslinked human, bovine, equine, ovine, caprine, porcine, feline or canine tissue. In some embodiments, the sheet of flexible material comprises a flexible polymer that comprises a woven polymer, PTFE, polypropylene, polyester, a coated polymer material or a combination of one or more of the foregoing materials or a bioequivalent substitute for one or more of the foregoing materials. In some embodiments, the sheet of flexible comprises an resorbable or a non-resorbable material. Additionally, in some embodiments, the invention provides a surgical kit comprising a surgical instrument of the invention and a flexible implant of the invention. In some embodiments, the invention also provides an implantation device, comprising: a surgical instrument of the invention and a flexible implant of the invention, where the implant is attached to the surgical instrument.

The instrument of the invention may be adapted to be used in a variety of surgical procedures, including open, mini-open and arthroscopic surgery. Thus, in some embodiments, the invention provides a surgical method, comprising: (a) placing a flexible implant having at least one aperture on a surgical instrument of the invention; (b) placing the flexible implant in contact with a bodily tissue in a mammalian body; (c) securing the flexible implant to the bodily tissue; and removing the surgical instrument from the mammalian body. In some embodiments, placing the flexible implant in contact with the bodily tissue produces a construct comprising a bodily tissue layer sandwiched between two flexible implant layers. In some specific embodiments, the construct further comprises one or more sutures, staples, screws or other tissue attachment devices. In some embodiments, the invention also provides an implantation device, comprising a surgical instrument of the invention and a flexible implant of the invention attached to the instrument of the invention.

In some embodiments, the invention provides a method of using an implantation device of the invention, comprising: (a) placing the flexible implant in contact with a tissue within a mammalian body; (b) securing the flexible implant to the tissue; and (c) removing the instrument from the mammalian body. In some embodiments, the flexible implant is placed in contact with a tissue to form a construct comprising a bodily tissue layer sandwiched between two flexible implant layers. In some embodiments, the tissue within the mammalian body is a bodily tissue. In some embodiments, the construct comprises a living tissue layer between two flexible implant layers. In some embodiments, the construct further comprising staples, sutures, anchors or a combination of two or more of the foregoing. In some embodiments, the construct is located within a mammalian body. In some embodiments, the construct is located with a human, bovine, equine, ovine, caprine, porcine, feline or canine body. In some embodiments, the human, bovine, equine, ovine, caprine, porcine, feline or canine body is a living human, bovine, equine, ovine, caprine, porcine, feline or canine body. In some embodiments, the invention provides a flexible implant comprising a flexible sheet having at least one tab having an aperture through the tab or having scoring adapted to permit removal of a chad from the tab to form an aperture. In this context, the term chad is that portion of the tab that, once removed, leaves in its place an aperture according to the invention. In some embodiments, the flexible sheet comprises soft tissue or a flexible polymer. In some embodiments, the flexible sheet is a soft tissue comprising a crosslinked mammalian tissue, such as crosslinked human, bovine, equine, ovine, caprine, porcine, feline or canine tissue. In some embodiments, the flexible sheet comprises a flexible polymer that comprises a woven polymer, PTFE, polypropylene, polyester, a coated polymer material or a combination of one or more of the foregoing materials or a bioequivalent substitute for one or more of the foregoing materials. In some embodiments, the flexible sheet comprises an resorbable or a non-resorbable material.

Additional needs met, and additional features and advantages provided, by the present invention will become apparent to the person having skill in the art upon consideration of the description herein as well as the appended drawings and claims.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a perspective view of a surgical instrument of the present invention.

FIG. 2 is a close-up view of the jaws of a surgical instrument of the present invention.

FIG. 3 is a perspective view of an implantation device of the present invention, comprising a surgical instrument of the invention and a flexible implant of the invention, as well as end of a tissue to which the flexible implant is to be attached.

FIG. 4 is a top view of a flexible implant of the present invention.

FIG. 5 is a side cutaway view of the jaws of a surgical instrument of the invention holding a flexible implant in place on the surface of a tissue to which the flexible implant is to be attached.

FIG. 6 is a perspective view of a surgical instrument of the present invention having an alternative head configuration.

FIG. 7 is a close-up view of an alternative jaw configuration for a surgical instrument according to the invention.

FIGS. 8A and 8B are straight and curved handles, respectively, of an instrument of the invention. The angle β is the angle of the handle curves.

FIGS. 9A, 9B, 9C and 9D are four different jaw configurations for surgical instruments of the invention.

FIGS. 10A and 10B are top-down views of instruments of the invention having bent heads. The angle α is the angle that the jaw makes with the arm to which it is attached.

### DETAILED DESCRIPTION OF THE INVENTION

The invention provides a surgical instrument (or “instrument”) for manipulating, placing and holding in place flexible implants during implantation surgery. The surgical instrument comprises a set of jaws having on at least one of the jaws at least one implant hook. Each implant hook is adapted to hold a flexible implant so that it may be manipulated, clamped in place and attached to a bodily tissue during implantation surgery.

In some embodiments, the invention provides a surgical instrument having two or more implant hooks. Thus, some embodiments of the invention provide a surgical instrument comprising a distal end and a proximal end, the distal end comprising a first implant hook and a second implant hook; and said proximal end comprising an actuator, said first and second implant hooks being moveable toward and away from one another by manipulation of said actuator. In some embodiments, the surgical instrument is adapted, e.g. in size and arrangement of the jaws, for arthroscopic surgery. In some embodiments, the actuator is a pair of handles, although other types of actuators are known in the art and may be used in place of handles. The handles may be straight or may be bent or curved. In some embodiments, the actuator is a thumb actuator. In some preferred embodiments, the distal end further comprises at least one textured contact pad or grip. In particular embodiments, each grip is located on a contact pad located on the inside of the jaws. The contact pads or grips are adapted to securely hold the jaws in place on the bodily tissue when the jaws are closed and preferably locked in place. In some preferred embodiments, at least two jaws comprise textured grips. In some preferred embodiments, at least two such grips are located on opposing jaws.

In further embodiments, the surgical instrument of the present invention comprises: (a) a first arm having a distal end and a proximal end, the distal end having at least one implant hook; and (b) a second arm having a distal end and a proximal end, the distal end having a jaw, which optionally possesses at least one implant hook, wherein the first and second arms are attached to one another by a pivot. In some preferred embodiments, the proximal end of the surgical instrument further comprises handles. In particular embodiments, the surgical instrument has a distal end that comprises at least two opposed pairs of implant hooks. In some such embodiments, the surgical instrument comprises at least 2, at least 4, at least 6 or at least 8 hooks, i.e. from 2 to 10, from 2 to 8 or from 4 to 6. In some embodiments, the implant hooks of the surgical instrument further comprise textured grips. In particular embodiments, the implant hooks further comprise at least one textured grip, at least two textured grips, from 1 to 10 textured grips, from about 2 to 8 textured grips, from about 4 to about 8 textured grips or about 4 to about 6 textured grips. In some embodiments, the surgical instrument comprises at least one grasping slit formed between a hook and an overhang. Each grasping slit (or simply “slit”) allows a portion of a flexible implant to slide within the slit where it is held in place while a surgeon places the flexible implant within the body of a surgical subject and attaches the flexible implant within the subject. In particular embodiments, the surgical instrument comprises at least one grasping slit, at least two grasping slits, from 1 to 10 grasping slits, from about 2 to 8 grasping slits, from about 4 to about 8 grasping slits or about 4 to about 6 grasping slits. In some preferred embodiments, the number of textured grips equals the number of grasping slits. In some embodiments, the numbers of textured grips and grasping slits are different.

The invention further provides a flexible implant, which comprises a sheet of biocompatible flexible material for implantation having at least two apertures of a size appropriate to rest on implant hooks. In some embodiments, the flexible material is a mammalian soft tissue, especially a cured or crosslinked mammalian tissue. In particular embodiments, the flexible material is a crosslinked mammalian tissue selected from crosslinked human, crosslinked bovine, crosslinked equine, crosslinked ovine, crosslinked caprine, crosslinked feline or crosslinked canine tissue. The crosslinked tissue include glutaraldehyde or carbodiimide crosslinked tissues. The methods for preparing crosslinked biological tissues are known in the art. In particular, some methods for crosslinking and sterilizing tissues for transplantation are included in U.S. Pat. Nos. 6,521,179; 6,506,339; 5,911,951; 5,733,339; and 5,447,536, which are incorporated herein by reference. The particular tissue used may include pericardium, dermis, fascia, delaminated intestine (SIS—small intestinal sub-mucosa) or reconstituted collagen or other biocompatible tissue. In particular embodiments, the particular tissue is fetal bovine dermis or reconstituted collagen. In other particular embodiments, the tissue is carbodiimide crosslinked equine pericardium or porcine pericardium. In some embodiments, the flexible implant is made from a biocompatible polymer, such as polytrifluroethylene (PTFE), polypropylene, polyester, a coated polymer material or a combination of natural and/or synthetic materials.

The invention also provides a surgical kit comprising a surgical instrument of the invention having at least one implant hook and a flexible implant comprising a sheet of soft tissue having at least one aperture adapted to engage said implant hooks. In some embodiments, the implant device has one hook only. In some embodiments, the implant device has two or more hooks. The two or more hooks may be located on either jaw; although in some preferred embodiments, at least one hook is located on each jaw. In some such embodiments, the implant device further includes one or more members of the group consisting of: contact pads, fingers, grasping slits, suture or needle channels, hollow needles and puncture spikes. In some preferred embodiments, each implant hook is augmented with a grasping slit. In some such embodiments, the surgical instrument includes two or more members of the group consisting of: contact pads, fingers, grasping slits, suture or needle channels, hollow needles and puncture spikes. In some preferred embodiments, each implant hook is augmented with a grasping slit.

The invention further provides a surgical method comprising: placing a flexible implant comprising a sheet of flexible material on a surgical instrument having at least one implant hook; placing the flexible implant in contact with a bodily tissue in a mammalian body; securing the flexible implant to the bodily tissue; and removing the surgical instrument from the mammalian body. The flexible implant is described in more detail above, as is the surgical instrument. The bodily tissue within a mammalian body is a tissue that can have a flexible implant applied to it; in some preferred embodiments it is a connective tissue, such as a tendon or a ligament, although it may also be a blood vessel, a bone, an organ or other bodily tissue.

The invention further provides as an article of manufacture a surgical implantation device comprising a surgical instrument according to the present invention combined with a flexible implant according to the present invention. Thus, in some embodiments, the invention provides a surgical instrument comprising a set of jaws in communication with an actuator, said set of jaws comprising at least one implant hook and said actuator adapted to open and close said jaws in combination with a flexible implant comprising a sheet of flexible material having at least one aperture adapted to be engaged by an implant hook. In some embodiments, the invention provides a surgical implantation device comprising: (a) a surgical instrument comprising a distal end and a proximal end, the distal end comprising a first implant hook and a second implant hook and said proximal end comprising an actuator, said first and second implant hooks being moveable toward and away from one another by manipulation of said actuator; and (b) a flexible implant comprising a sheet of flexible material having at least a first aperture and a second aperture, wherein the two apertures are adapted to be engaged by said implant hooks of said surgical instrument. In some embodiments, the method comprises placing the flexible implant in contact with a living tissue within a mammalian body; securing the flexible implant to the living tissue; and removing the surgical instrument from the mammalian body.

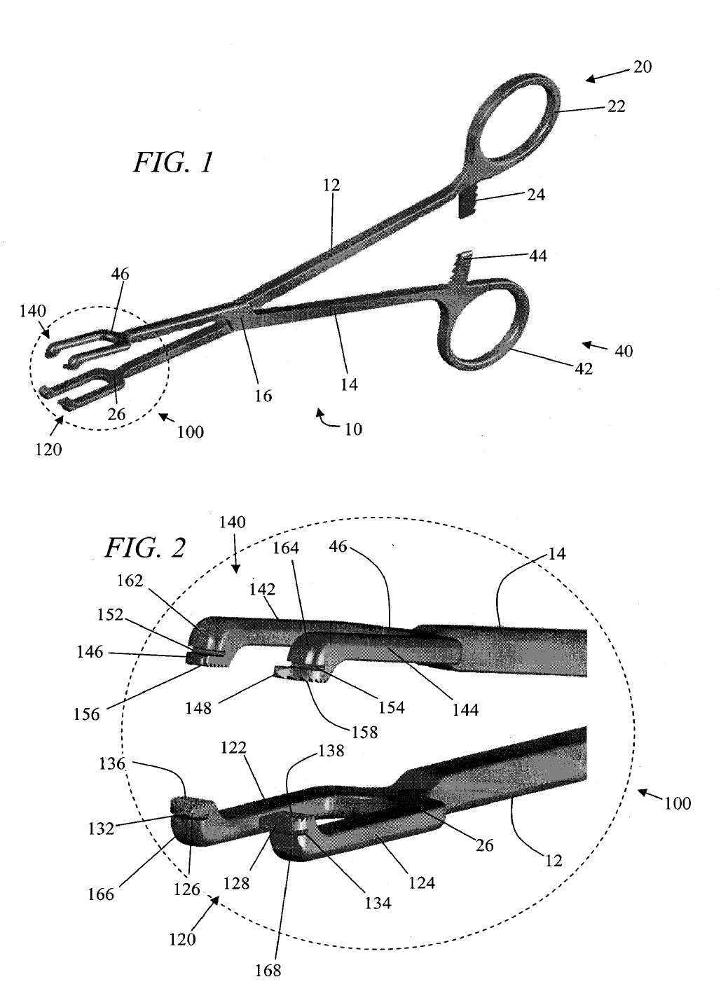

Various views of some embodiments of the invention are shown in FIGS. 1-10B. The view shown in FIG. 1 is a perspective of an entire surgical instrument 10, which is one embodiment of the invention, while the view in FIG. 2 is a zoom view of the jaws 100 of the surgical instrument 10. The surgical instrument 10 comprises a first arm 12 and a second arm 14, which are joined to one another at a pivot point 16, about which arms 12 and 14 pivot. The first arm 12 has a proximal end 20 and a distal end 120. The second arm 14 has a proximal end 40 and a distal end 140. Movement of the proximal end 40 of the second arm 14 toward the proximal end 20 of the first arm 12 causes the distal ends 120 and 140 to move toward one another, and that movement of the proximal ends 20 and 40 away from one another causes the distal ends 120 and 140 to move away from one another.

As can be seen in FIG. 1, the surgical instrument 10 has jaws 100 comprising a lower jaw 26 at the distal end 120 of the first arm 12, and an upper jaw 46 at the distal end 140 of the second arm 14. The space between the jaws 26 and 46 is referred to herein as the interior (or inside) of the jaws. The jaws 100 can be seen more clearly in FIG. 2, where it can be seen that upper jaw 46 is forked, having a first finger 142, which terminates in a first hook 146, and a second finger 144, which terminates in a second hook 148. A slit 152 is formed between the hook 146 and an overhang 162. Likewise, finger 144 has a hook 148 at its distal end; and a slit 154 is formed between the hook 148 and an overhang 164. The slits 152, 154 are adapted to receive and hold a portion of a flexible implant, such as those of the invention. Hook 146 also has a contact pad 156 on the side opposite the slit 152. Likewise, hook 148 has a contact pad 158 on the side opposite the slit 154. The contact pads 156 and 158 are textured, in this case serrated, in the depicted embodiment, in order to aid the hooks 146, 148 in gripping bodily tissues and/or implants during the surgical procedure. In some embodiments, the contact pads may be smooth or may have other types of texturing, such as stippling or cross-hatching, to provide a similar effect. In some embodiments, the contact pads may have channels for passage of needles and/or sutures, or they may have other features such as hollow needles or puncture spikes. In some embodiments, at least one contact pad has two or more features such as texturing, stippling, cross-hatching, a hollow needle, a channel (such as a needle channel or a suture channel), or a puncture spike.

In the depicted embodiment, the lower jaw 26 has a first finger 122, which terminates in a first hook 126, and a second hook 128. A slit 132 is formed between the hook 126 and an underhang 166. In turn, finger 124 has a hook 128 at its distal end; and a slit 134 is formed between the hook 128 and an under-hang 168. Hook 126 also has a contact pad 136 on the side opposite the slit 132. Likewise, hook 128 has a contact pad 138 on the side opposite the slit 134. The contact pads 136 and 138 are textured (serrated) in the depicted embodiment, in order to aid the hooks in gripping bodily tissues and/or implants during a surgical procedure. In some embodiments, the contact pads may be smooth or may have other types of texturing, such as stippling or cross-hatching, to provide a similar effect. In some embodiments, the contact pads may have channels for passage of needles and/or sutures, or they may have other features such as hollow needles or puncture spikes. In some embodiments, at least one contact pad has two or more features such as texturing, stippling, cross-hatching, a hollow needle, a channel (such as a needle channel or a suture channel), or a puncture spike.

While the jaws 100 are shown having two fingers on each of the upper jaw 46 and the lower jaw 26, the person skilled in the art will recognize that any number of fingers can be employed in the jaw structure. Suitable numbers of fingers on each jaw include from 1 to 10, preferably from 1-5, more preferably 1, 2, 3 or 4. The number of fingers on each jaw may be the same or different. Thus, one jaw may have 1, 2, 3, 4, 5, 6, 7, 8, 9, 10 or more fingers and the other jaw may independently have 1, 2, 3, 4, 5, 6, 7, 8, 9, 10 or more fingers. In some preferred embodiments, the upper and lower jaws have the same number of fingers. In particularly preferred embodiments, each jaw has 2, each jaw has 3, each jaw has 4 or each jaw has five or more fingers. While the depicted embodiment has an equal number of opposed fingers and contact pads, other embodiments may employ an offset pattern such that the contact pads of one jaw are not flush with the contact pad on the other jaw when the jaws are closed and such embodiments are considered included within the present invention. The person skilled in the art will know how to prepare such embodiments by making the appropriate modifications to the depicted embodiment.

The first arm 12 has a handle 22 formed in the proximal end 20 of the arm 12. The second arm 14 has a handle 42 formed in the proximal end 40 of the arm 14. The surgeon may use these handles 22 and 42 by placing a thumb and a finger through the handles 22 and 42 and moving them toward and away from one another. Thus, in some embodiments of the invention, the actuator is a scissor-like pair of handles attached to arms that are adapted to move the jaws of the surgical instrument relative to one another. The scissor-like opening and closing of clamp devices is know to those skilled in the art. Alternative actuators, such as thumb-type actuators are also know in the art and may be substituted for the depicted handle-type actuator in some embodiments of the invention.

The arms 12 and 14 also have ratchet locks 24 and 44, respectively, located near the proximal ends 20, 40, thereof. Together the ratchet locks 24 and 44 allow the surgeon to lock the instrument in a particular position. The operation of such ratchet hooks is known to those skilled in the art. Other locking devices known in the art may be substituted for the depicted locking device in some embodiments of the invention.

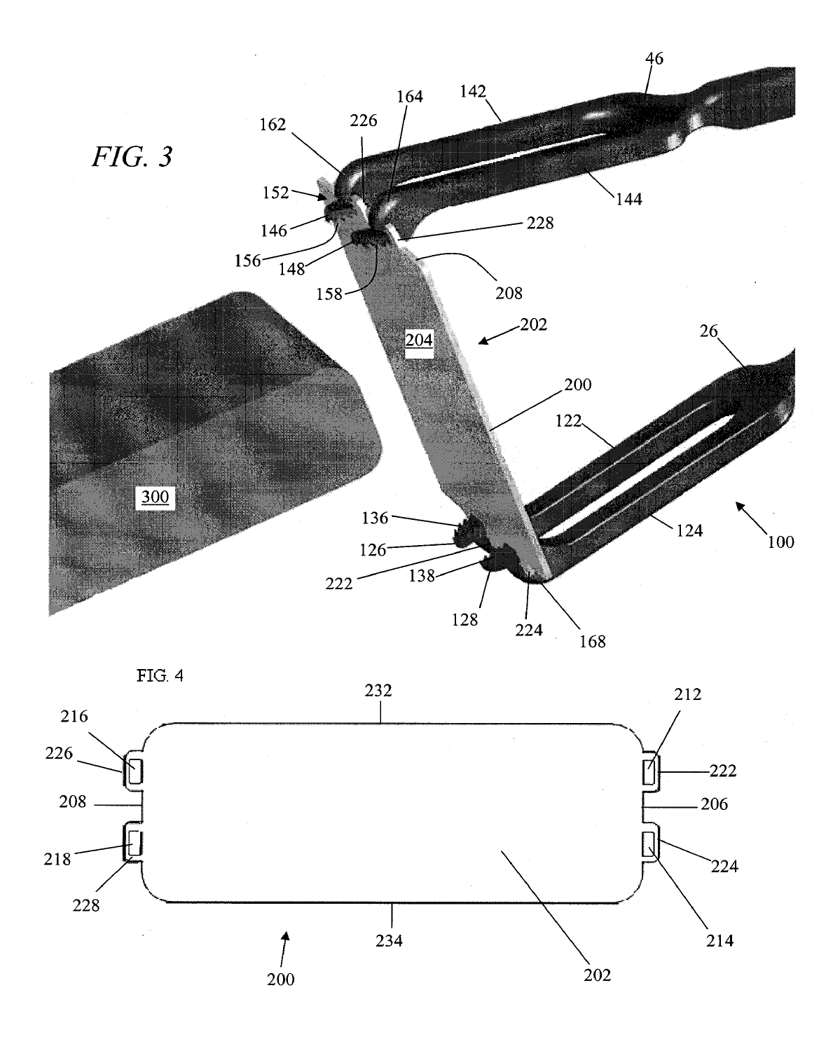

The operation of the surgical instrument according to the invention provides useful advantages to a surgeon implanting a flexible implant into a subject. In general, such operation is aided by use of a specially tailored flexible implant as depicted in FIG. 4. The flexible implant 200 comprises a sheet of biological tissue having a top side 202, a bottom side 204 (visible in FIG. 3), right edge 206, left edge 208, top edge 232 and bottom edge 234. (The terms top, bottom, left and right are assigned arbitrarily and are for purposes of orientation only.) Four tabs 222, 224, 226 and 228 are arranged along the right edge 206 and left 208 of the flexible implant 200. As depicted, the flexible implant has two tabs 222 and 224 on the right edge 206 and two tabs 226 and 228 on the left edge 208; however other arrangements are possible, as discussed in more detail below. Each of tabs 222, 224, 226 and 228 has an aperture 212, 214, 216 and 218, respectively, which is made of an appropriate size to fit over the end of a flexible implant hook on the surgical instrument according to the invention. In general, the flexible implant will have at least as many tabs as there are flexible implant hooks on the surgical instrument with which it is to be used; and in some preferred embodiments the tissue has the same number of tabs as the number of flexible implant hooks on the instrument with which it is to be used. Thus, in some embodiments the number of tabs on each edge of the flexible implant is from 1 to 10, preferably from 1 to 5, more preferably 1, 2, 3 or 4. The number of tabs on each edge of the flexible implant may be the same or different. Thus, one edge may have 1, 2, 3, 4, 5, 6, 7, 8, 9, 10 or more tabs and the opposite edge may independently have 1, 2, 3, 4, 5, 6, 7, 8, 9, 10 or more tabs. While the embodiment shown has an aperture in each tab, and it is considered preferable to have the apertures formed during the manufacturing process of the flexible implant, it is possible for the apertures to be formed by surgical personnel prior to, or while, placing the flexible implant on the surgical instrument. In some embodiments, the positions where the apertures are to be formed are perforated so that the apertures can be formed by punching out the chads formed by the perforations. These chads can easily be removed from the tabs by surgical personnel before or during placement of the flexible implant on the surgical instrument. Moreover, while the tabs are depicted as pronounced protrusions from the ends of the flexible implant, it is also possible for the tabs to much less pronounced protrusions or to be flush with the ends of the flexible implant. Additionally, while the tabs are depicted as being on the short edges 206, 208 of the flexible implant 200, they can easily be formed on the long edges 232, 234 of the flexible implant 200, depending upon the particular surgical procedure being employed.

The flexible implant 200 is depicted in FIG. 3 in conjunction with the surgical instrument 10. It can be seen from FIG. 3 that the flexible implant 200 hangs on the flexible implant hooks 126, 128, 146 and 148 by the soft tissue tabs 222, 224, 226 and 228, respectively, so that the bottom side 204 of the flexible implant 200 faces the end of the tendon 300.

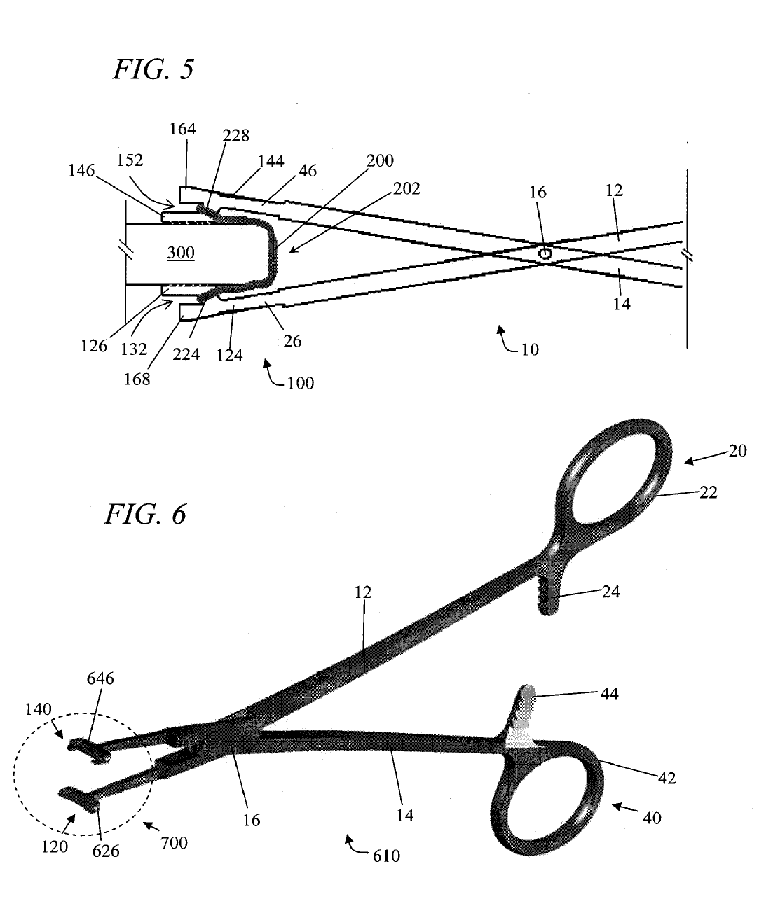

As shown in FIG. 5, which is a cutaway side view of the instrument 10, tissue implant 200 and tendon 300, closure of the jaws 100 on the end of the tendon 300 causes the flexible implant 200 to be held securely against the tendon 300. The operator, such as a surgeon or surgical assistant, can lock the jaws in place using the ratchet 24, 44 depicted in FIG. 1. This allows the instrument 10 to be left in place, securely holding the flexible implant 200 in place while the remainder of the procedure is carried out. After the flexible implant 200 has been attached to the tendon 300, the surgical instrument may be removed from the flexible implant 200, leaving it in place within the body.

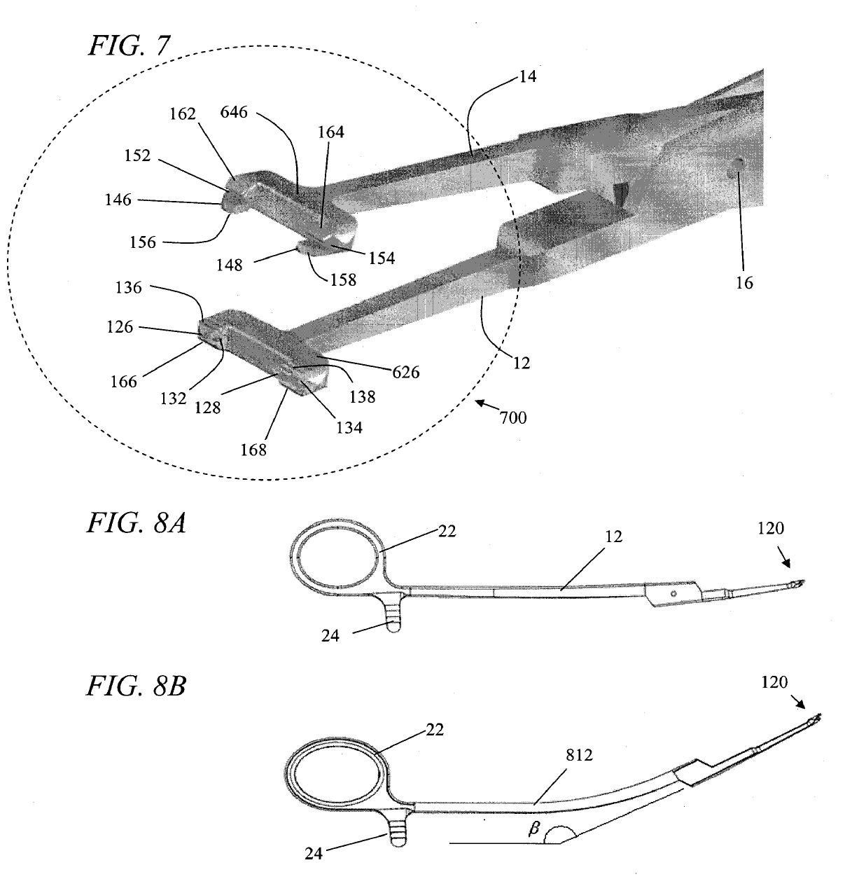

Another embodiment of the invention is depicted in FIGS. 6 and 7. The view shown in FIG. 6 is a perspective of an entire surgical instrument 610, which is one embodiment of the invention, while the view in FIG. 7 is a zoom view of the jaws 700 of the surgical instrument 610. The surgical instrument 610 comprises a first arm 12 and a second arm 14, which are joined to one another at a pivot point 16, about which arms 12 and 14 pivot. The first arm 12 has a proximal end 20 and a distal end 120. The second arm 14 has a proximal end 40 and a distal end 140. Movement of the proximal end 40 of the second arm 14 toward the proximal end 20 of the first arm 12 causes the distal ends 120 and 140 to move toward one another, and that movement of the proximal ends 20 and 40 away from one another causes the distal ends 120 and 140 to move away from one another. Thus, in this as in some other embodiments of the invention, the actuator is a scissor-like pair of handles adapted to move the jaws of the surgical instrument relative to one another.

As can be seen in FIG. 6, the surgical instrument 610 has jaws 700 comprising a lower jaw 626 at the distal end 120 of the first arm 12, and an upper jaw 646 at the distal end 140 of the second arm 14. The space between the jaws 626 and 646 is referred to herein as the interior (or inside) of the jaws. The jaws 700 can be seen more clearly in FIG. 7, where it can be seen that upper jaw 646 terminates in a first hook 146 and a second hook 148. A slit 152 is formed between the hook 146 and an overhang 162. A second slit 154 is formed between the hook 148 and an overhang 164. The slits 152, 154 are adapted to receive and hold a portion of a flexible implant, such as those of the invention. Hook 146 also has a contact pad 156 on the side opposite the slit 152. Likewise, hook 148 has a contact pad 158 on the side opposite the slit 154. The contact pads 156 and 158 are textured, in this case serrated, in the depicted embodiment, in order to aid the hooks 146, 148 in gripping bodily tissues and/or implants during the surgical procedure. In some embodiments, the contact pads may be smooth or may have other types of texturing, such as stippling or cross-hatching, to provide a similar effect. In some embodiments, the contact pads may have channels for passage of needles and/or sutures, or they may have other features such as hollow needles or puncture spikes. In some embodiments, at least one contact pad has two or more features such as texturing, stippling, cross-hatching, a hollow needle, a channel (such as a needle channel or a suture channel), or a puncture spike.

In the embodiment depicted in FIGS. 6 and 7, the lower jaw 626 terminates in a first hook 126, and a second hook 128. A slit 132 is formed between the hook 126 and an under-hang 166. A slit 134 is formed between the hook 128 and an under-hang 168. Hook 126 also has a contact pad 136 on the side opposite the slit 132. Likewise, hook 128 has a contact pad 138 on the side opposite the slit 134. The contact pads 136 and 138 are textured (serrated) in the depicted embodiment, in order to aid the hooks in gripping biological tissues and/or flexible implants during the surgical procedure. In some embodiments, the contact pads may be smooth or may have other types of texturing, such as stippling or cross-hatching, to provide a similar effect. In some embodiments, the contact pads may have channels for passage of needles and/or sutures, or they may have other features such as hollow needles or puncture spikes. In some embodiments, at least one contact pad has two or more features such as texturing, stippling, cross-hatching, a hollow needle, a channel (such as a needle channel or a suture channel), or a puncture spike.

While the jaws 700 are shown having two implant hooks on each of the upper jaw 646 and the lower jaw 626, the person skilled in the art will recognize that any number of implant hooks can be employed in the jaw structure. Suitable numbers of implant hooks on each jaw include from 1 to 10, preferably from 1-5, more preferably 1, 2, 3 or 4. The number of implant hooks on each jaw may be the same or different. Thus, one jaw may have 1, 2, 3, 4, 5, 6, 7, 8, 9, 10 or more implant hooks and the other jaw may independently have 1, 2, 3, 4, 5, 6, 7, 8, 9, 10 or more implant hooks. In some preferred embodiments, the upper and lower jaws have the same number of implant hooks. In particularly preferred embodiments, each jaw has 2, each jaw has 3, each jaw has 4 or each jaw has five or more implant hooks. While the embodiment in FIGS. 6 and 7 has an equal number of opposed implant hooks and contact pads, other embodiments may employ an offset pattern such that the contact pads of one jaw are not flush with the contact pad on the other jaw when the jaws are closed and such embodiments are considered included within the present invention. The person skilled in the art will know how to prepare such embodiments by making the appropriate modifications to the depicted embodiment.

The first arm 12 has a handle 22 formed in the proximal end 20 of the arm 12. The second arm 14 has a handle 42 formed in the proximal end 40 of the arm 14. The surgeon may use these handles 22 and 42 by placing a thumb and a finger through the handles 22 and 42 and moving them toward and away from one another. Thus, in some embodiments of the invention, the actuator is a scissor-like pair of handles attached to arms that are adapted to move the jaws of the surgical instrument relative to one another. The scissor-like opening and closing of clamp devices is know to those skilled in the art. Alternative actuators, such as thumb-type actuators are also know in the art and may be substituted for the depicted handle-type actuator in some embodiments of the invention.

The arms 12 and 14 also have ratchet locks 24 and 44, respectively, located near the proximal ends 20, 40, thereof. Together the ratchet locks 24 and 44 allow the surgeon to lock the clamp in a particular position. The operation of such ratchet hooks is known to those skilled in the art. Other locking devices known in the art may be substituted for the depicted locking device in some embodiments of the invention.

The jaws of the surgical instrument of the invention may take on a variety of alternative configurations depending upon the particular procedure for which the surgical instrument is to be used. For example, the arms of the actuator may be straight or curved. In FIG. 8A there is depicted a straight actuator arm 12 comprising a handle 22, a lock 24 and a jaw 120. In FIG. 8B, in contrast, there is depicted a curved arm 812, having handle 22 and lock 24 at one end and jaw 120 at the other end. The arm 812 is bent through angle β. In some particular embodiments, β is about 5 to about 45 degrees. In other particular embodiments, the β is about 5, about 10, about 15, about 20, about 25, about 30, about 35, about 40, about 45, about 50, about 65, about 70, about 75, about 80, about 85, about 90, about 95 or about 100 degrees.

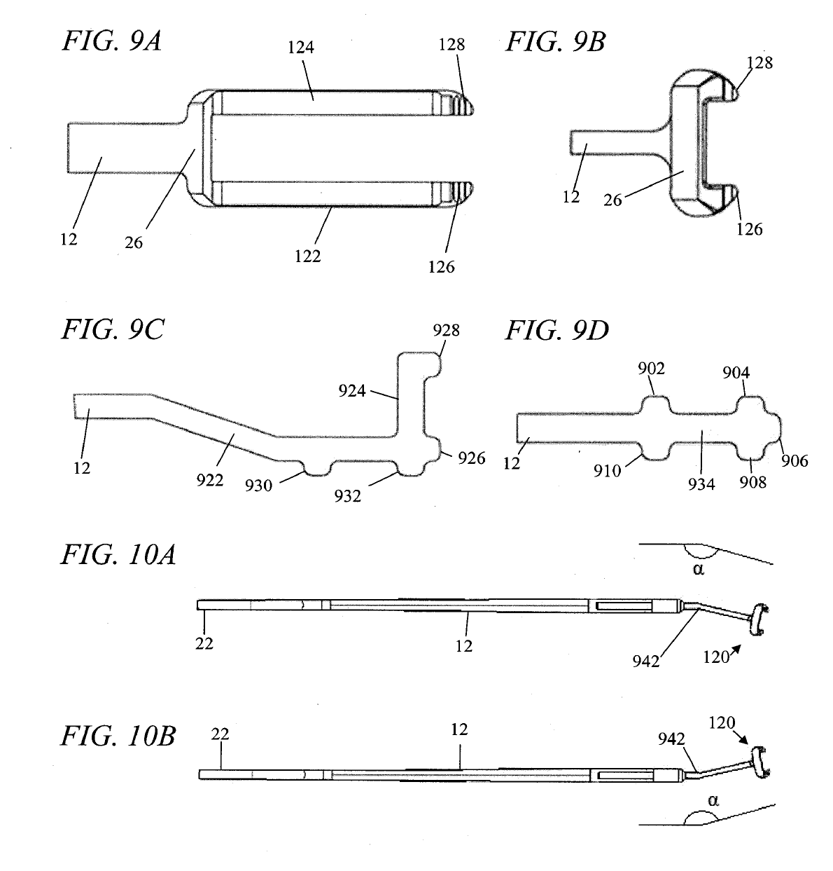

In addition, the jaws of the surgical instrument may take on a variety of different forms. For example, in FIG. 9A, there is depicted a U-shaped jaw structure in which the arm 12 terminates in a jaw 26 having a pair of fingers 122 and 124, each of which has formed at its end an implant hook 126, 128 according to the invention. In FIG. 9B, there is depicted a T-shaped jaw in which the arm 12 terminates in a jaw 26 having a pair of implant hooks 126, 128. In FIG. 9C, a so-called L-tip is depicted, in which the arm 12 terminates in a bent finger 922 having formed at its end a hook 926; an extension 924 juts out from the end of the finger 922 at an angle and has at its end a second hook 928. Another hook 930 juts out of the side of the bent finger 922. Yet another hook 932 juts out the side of the bent finger 922 making an approximately right angle to the hook 926 on the end of the bent finger 922. In alternate embodiments, one or more hooks 926, 928, 930 or 932 may be absent. In still further embodiments, additional hooks may be added to accommodate differently configured flexible implants. In FIG. 9D, a so-called I-tip is depicted. The arm 12 terminates in a finger 934 having an implant hook 906 formed in its end and a plurality of additional implant hooks 902, 904, 908 and 910 formed in its sides. Although not specifically depicted in these views, in embodiments of the invention, the fingers 122, 124, 932 and 932 and extension 924 have formed at their ends one or more grasping slits and/or contact pads as well as other features, such as channels, hollow needles and/or spikes.

The head of the surgical instrument may also be bent up, down, or to either side in order to adapt the device to working in a variety of bodily spaces. In FIGS. 10A and 10B a pair of head configurations is depicted. The arm 12 is bent at point 942 at an angle α with respect to the rest of the arm 12 so that jaw 120 is at an angle with respect to the handle 22. As can be seen from FIGS. 10A and 10B the head may be bent to either side. Although not specifically depicted in these views, the jaws may also have formed at their ends grasping slits and/or contact pads as well as other features, such as channels, punches and/or hollow needles. The angle α may be any convenient angle necessary for manipulating and clamping the instrument in the bodily space for which it is designed. In some embodiments, α is from about 1 to about 135 degrees. In some particular embodiments, α is about 30 to about 90 degrees. In other particular embodiments, the α is about 5, about 10, about 15, about 20, about 25, about 30, about 35, about 40, about 45, about 50, about 65, about 70, about 75, about 80, about 85, about 90, about 95 or about 100 degrees.

The surgical instrument according to the invention may be used to attach flexible implants to a variety of tissues within the body. In some embodiments, the flexible implant is used to clamp a reinforcing or connecting tissue implant to a tendon or ligament while the surgeon attaches the implant to the tendon or ligament using suitable attachment devices, such as sutures or staples. Other uses and advantages of the surgical instruments according to the invention may become apparent to the person skilled in the art upon consideration of the invention described herein, and such uses and advantages are considered to be within the scope of the present invention.

The flexible implants according to the invention are advantageously used with surgical instruments according to the invention, e.g. to repair injured tendon or ligament or as reinforcement for such tissues, especially as tissue reinforcement when connecting ruptured ligament or tendon to bone. However, other uses and advantages of the surgical instrument according to the invention may become apparent to the person skilled in the art upon consideration of the invention described herein, and such uses and advantages are considered to be within the scope of the present invention.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A surgical instrument comprising a set of jaws in communication with an actuator, said set of jaws comprising at least one implant hook and said actuator adapted to open and close said jaws.

2. The surgical instrument of claim 1, wherein the set of jaws comprises only one implant hook.

3. The surgical instrument of claim 1, wherein the set of jaws comprises two or more implant hooks.

4. The surgical instrument of claim 1, wherein the set of jaws comprises 2, 3, 4, 5, 6, 7, 8, 9, 10 or more implant hooks.

5. The surgical instrument of claim 1, wherein the set of jaws further comprises at least one contact pad, finger, grasping slit, suture or needle channel, hollow needle or puncture spike.

6. The surgical instrument of claim 1, wherein the set of jaws further comprises at least one contact pad.

7. The surgical instrument of claim 1, wherein the set of jaws further comprises two or more contact pads.

8. The surgical instrument of claim 1, wherein the set of jaws comprises 2, 3, 4, 5, 6, 7, 8, 9, 10 or more contact pads.

9. The surgical instrument of claim 1, wherein the set of jaws further comprises at least one grasping slit.

10. The surgical instrument of claim 1, wherein the set of jaws comprises only one grasping slit.

11. The surgical instrument of claim 1, wherein the set of jaws comprises 2, 3, 4, 5, 6, 7, 8, 9, 12 or more grasping slits.

12. The surgical instrument of claim 1, wherein the set of jaws is disposed at an angle with respect to the actuator.

13. The surgical instrument of claim 12, wherein the angle is about 1 to about 135 degrees.

14. The surgical instrument of claim 13, wherein the angle is about 30 to about 90 degrees.

15. The surgical instrument of claim 13, wherein the angle is about 5, about 10, about 15, about 20, about 25, about 30, about 35, about 40, about 45, about 50, about 65, about 70, about 75, about 80, about 85, about 90, about 95 or about 100 degrees.

16. The surgical instrument of claim 1, wherein the set of jaws comprises at least two jaws, each jaw comprising at least one finger, at least one of said fingers having an implant hook thereon.

17. The surgical instrument of claim 16, wherein each jaw comprises one finger only.

18. The surgical instrument of claim 16, wherein each jaw independently comprises two or more fingers.

19. The surgical instrument of claim 16, wherein each jaw independently comprises 2, 3, 4, 5 or more fingers.

20. The surgical instrument of claim 16, wherein each jaw has the same number of fingers.

21. The surgical instrument of claim 20, wherein each finger on one jaw is directly opposed by a finger on the other jaw.

22. The surgical instrument of claim 16, wherein the two jaws have different numbers of fingers.

23. The surgical instrument of claim 22, wherein at least one finger on each jaw is directly opposed by at least one finger on the other jaw.

24. The surgical instrument of claim 22, wherein at least one finger on one jaw is offset with respect to the fingers on the opposing jaw.

25. The surgical instrument of claim 22, wherein all fingers on each jaw are offset with respect to the fingers on the other jaw.

26. The surgical instrument of claim 1, wherein at least one jaw comprises texturing, a needle or suture channel, a hollow needle or a puncture spike.

27. The surgical instrument of claim 26, wherein one jaw comprises a needle, a suture, a channel, a hollow needle or a puncture spike.

28. The surgical instrument of claim 26, wherein at least one jaw comprises texturing.

29. The surgical instrument of claim 26, wherein each jaw comprises texturing.

30. The surgical instrument of claim 26, wherein at least one jaw comprises a contact pad having texturing.

31. The surgical instrument of claim 26, wherein each jaw comprises a contact pad having texturing.

32. The surgical instrument of claim 1, wherein at least one jaw is a U-shaped or T-shaped.

33. The surgical instrument of claim 1, wherein at least one jaw comprises an I-tip or L-tip.

34. The surgical instrument if claim 1, wherein both jaws are U-shaped, both jaws are T-shaped, both jaws comprise an L-tip or both jaws comprise an I-tip.

35. The surgical instrument of claim 1, wherein the actuator further comprises a lock adapted to lock said jaws in a closed position.

36. The surgical instrument of claim 1, wherein the actuator comprises a set of handles or a thumb actuator.

37. The surgical instrument of claim 36, wherein the actuator is a set of handles.

38. The surgical instrument of claim 37, wherein the set of handles is curved.

39. The surgical instrument of claim 1, wherein the device is adapted for arthroscopic surgery.

40. The surgical instrument of claim 1, wherein the distal end further comprises at least one textured grip.

41. The surgical instrument of claim 1, wherein the first and second implant hooks further comprise textured grips.

42-53. (canceled)

54. A surgical kit, comprising:
a surgical instrument comprising a set of jaws in communication with an actuator, said set of jaws comprising at least one implant hook and said actuator adapted to open and close said jaws; and
a flexible implant comprising a sheet of flexible material having at least one aperture adapted to rest on an implant hook.

55-56. (canceled)

57. An implantation device, comprising:
a surgical instrument comprising a set of jaws in communication with an actuator, said set of jaws comprising at least one implant hook and said actuator adapted to open and close said jaws; and
a flexible implant, comprising a sheet of flexible material having at least one aperture adapted to rest on an implant hook, attached to said surgical instrument such that at least one aperture is held by at least one implant hook.

58-70. (canceled)
